(19) United States
(12) Patent Application Publication
(10) Pub. No.: US 2015/0012016 A1
(43) Pub. Date: Jan. 8, 2015

(54) SUTURE PASSER

(71) Applicant: Biomet Manufacturing, LLC, Warsaw, IN (US)

(72) Inventor: Kevin T. Stone, Winona Lake, IN (US)

(73) Assignee: Biomet Manufacturing, LLC, Warsaw, IN (US)

(21) Appl. No.: 14/322,698

(22) Filed: Jul. 2, 2014

Related U.S. Application Data
(60) Provisional application No. 61/842,652, filed on Jul. 3, 2013.

(51) Int. Cl. A61B 17/04

(52) U.S. Cl. CPC A61B 17/0469
USPC 606/144

(57) ABSTRACT

A suture passer for passing a suture through tissue during surgery includes a tissue grasper, a piercing member and a suture retriever. The tissue grasper has a first jaw with a first opening and a second jaw with a second opening. The first jaw and/or the second jaw is/are movable between an open position and a clamped position. The first opening and the second opening align relative to each other when in the clamped position. The first jaw has a suture positioner for positioning a suture across the first opening. The piercing member forms an opening in the tissue. The suture retriever includes a distal end and a hook positioned proximally of the distal end. The suture retriever passes at least partially through the first opening and the second opening and the hook is used to retrieve the suture held across the first opening.

### CROSS REFERENCE TO RELATED APPLICATIONS

This is a non-provisional application based upon U.S. Provisional Patent Application Ser. No. 61/842,652, entitled “SUTURE PASSER”, filed Jul. 3, 2013, which is incorporated herein by reference.

### BACKGROUND OF THE INVENTION

1. Field of the Invention

The present invention relates to surgical instruments, and, more particularly, to a surgical instrument used to create a hole in tissue and pass suture through the hole.

2. Description of the Related Art

Surgical procedures are often performed on a body, such as a human body, to repair or replace various portions thereof. For example, the soft tissues of the body may need to be reattached to bones or a tear in the soft tissue may need repaired due to trauma, overuse, surgical intervention, or disease.

Soft tissues can be reattached to a bone or repaired using fastening devices such as screws, staples and various types of suture anchors. Soft tissues are often fixed to various positions on the bone. For example, to replace a natural tendon fixation point or to replace the tendon itself, it may be desired to fix a graft to a selected bone area. One method of fixing a soft tissue to the selected area is to pass one end of a suture through a selected portion of the soft tissue to form a knot and fix another end of the suture to a selected area on the bone with a suture anchor.

Various suture passers have been created to pass suture through tissue. Current rotator cuff passers push suture up through the cuff and require a second instrument to pull the passed suture out of a separate portal. The separate portal must be properly aligned with the suture, and the suture must be positioned within the portal so that it can be retrieved by the separate instrument.

What is needed in the art is an easier way of passing suture through tissue and retrieving the suture.

### SUMMARY OF THE INVENTION

The present invention provides a suture passer which forms a hole in tissue, aligns the suture for retrieval, and retrieves the suture through the formed hole. The suture passer includes a tissue grasper and a suture retriever, separate from the tissue grasper, which captures the suture and can be used to directly shuttle the suture.

The invention in one form is directed to a suture passer for passing a suture through tissue during surgery, including a tissue grasper, a piercing member and a suture retriever. The tissue grasper has a first jaw with a first opening and a second jaw with a second opening. The first jaw and/or the second jaw is/are movable between an open position and a clamped position. The first opening and the second opening align relative to each other when in the clamped position. The first jaw has a suture positioner for positioning a suture across the first opening. The piercing member forms an opening in the tissue. The suture retriever includes a distal end and a hook positioned proximally of the distal end. The suture retriever passes at least partially through the first opening and the second opening and the hook is used to retrieve the suture held across the first opening.

The invention in another form is directed to a surgical kit for passing a suture through tissue during surgery, including a tissue grasper and a suture retriever. The tissue grasper has an elongate member with a first jaw and a second jaw positioned at a distal end of the elongate member. The first jaw has a first opening and the second jaw has a second opening. The first jaw and/or second jaw are movable between an open position and a clamped position. The first opening and the second opening align relative to each other when in the clamped position. The first jaw has a suture positioner for positioning a suture across the first opening. The suture retriever includes an elongate rod with a distal end, a piercing member at the distal end, and a hook positioned proximally of the piercing member.

An advantage of the present invention is that the hole can be formed in the tissue and the suture passed through the hole in a single operation.

Another advantage is that the tissue grasper holds and aligns the suture at a correct orientation so that it can be retrieved by the suture retriever.

Yet another advantage is that the tissue grasper is configured to easily align the suture retriever so that the suture can be reliably retrieved with little effort.

### BRIEF DESCRIPTION OF THE DRAWINGS

The above-mentioned and other features and advantages of this invention, and the manner of attaining them, will become more apparent and the invention will be better understood by reference to the following description of an embodiment of the invention taken in conjunction with the accompanying drawings, wherein:

FIG. 1 is a perspective view of a portion of an embodiment of a tissue grasper forming part the suture passer of the present invention;

FIG. 2 is another perspective view of the tissue grasper of FIG. 1;

FIG. 3 is another perspective view of the tissue grasper of FIGS. 1 and 2, with the jaws in an open position and a suture placed within one of the jaws;

FIG. 4 is another perspective view of the tissue grasper of FIG. 3;

FIG. 5 is a perspective view of the tissue grasper of FIGS. 1-4 loaded with suture and approaching rotator cuff tissue;

FIG. 6 is another perspective view of the tissue grasper of FIG. 5, gripping and manipulating the rotator cuff tissue;

FIG. 7 is a perspective view of a portion of the tissue grasper shown in FIGS. 1-6, and a portion of an embodiment of suture retriever forming part of the suture passer of the present invention;

FIG. 8 is another perspective view of a portion of the tissue grasper and suture retriever shown in FIG. 7, with the suture captured and pulled through the hole formed in the tissue;

FIG. 9 is another perspective view of a portion of the tissue grasper and suture retriever shown in FIGS. 7 and 8, with the suture shuttled out of the portal via the suture retriever;

FIG. 10 is another perspective view of a portion of the tissue grasper and suture retriever shown in FIGS. 8 and 9, with the tissue grasper unclamped from the tissue and the suture released from the tissue grasper; and

FIG. 11 is another perspective view of the tissue with the suture passed through the tissue.

Corresponding reference characters indicate corresponding parts throughout the several views. The exemplification set out herein illustrates an embodiment of the invention, and such exemplification is not to be construed as limiting the scope of the invention in any manner.

### DETAILED DESCRIPTION OF THE INVENTION

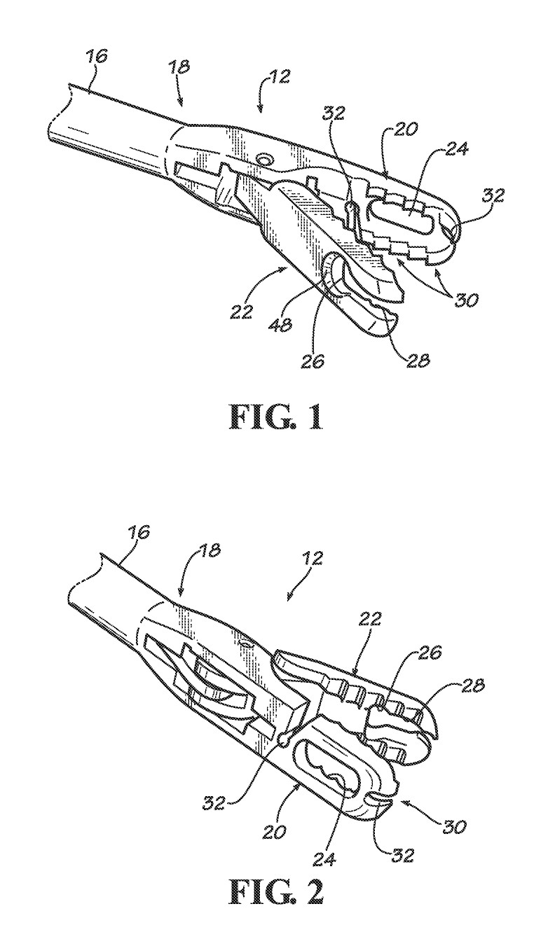

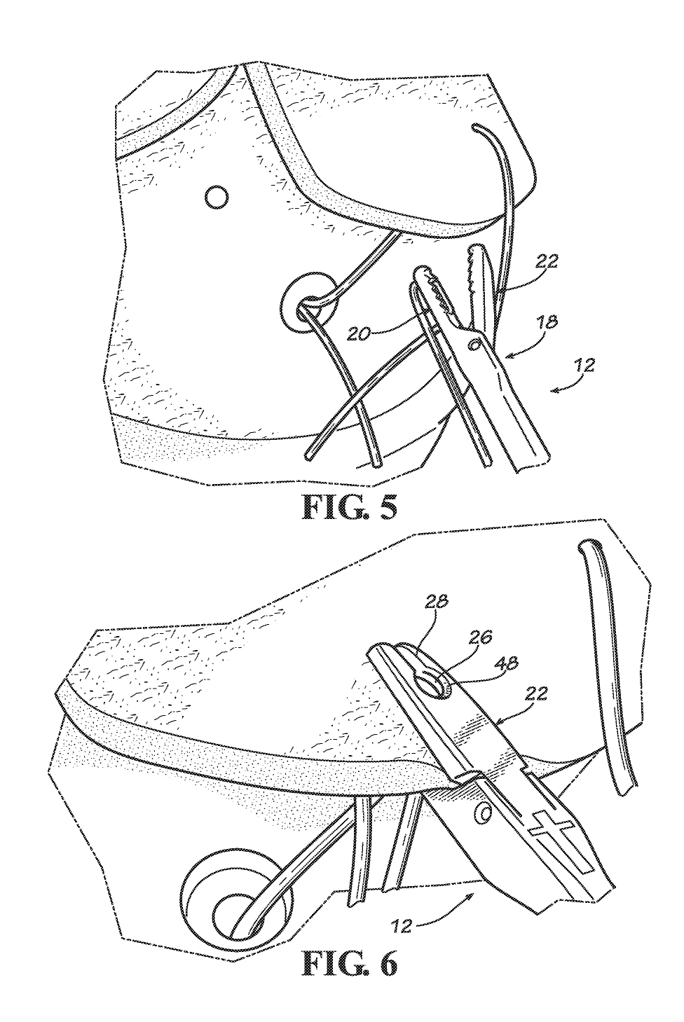

Referring now to the drawings, and more particularly to FIGS. 1-10, there is shown an embodiment of a suture passer 10 of the present invention which is used for passing suture through tissue during surgery. The suture passer 10 generally includes a tissue grasper 12 and a suture retriever 14. The term “suture”, as used herein, is intended broadly to mean any type of flexible elongate strand or element used during surgery to tie, attach, anchor, close, etc. tissue, such as a braided or monofilament suture, flexible wire, etc. In the illustrated embodiment, the tissue is in the form of a rotator cuff tissue, particularly a tendon, but could be a different type of tissue through which suture may be passed during surgery.

The tissue grasper 12 has a handle 16 with a distal end 18 to which is mounted a first jaw 20 and a second jaw 22. The first jaw 20 has a first opening 24 and the second jaw 22 has a second opening 26. The first jaw 20 and/or second jaw 22 are movable between an open position (FIGS. 1-5) and a clamped position (FIGS. 6-10). In the embodiment shown, the first jaw 20 is rigidly affixed to the handle 16, and the second jaw 22 is movable relative to the first jaw 20. However, the first jaw 20 can be the movable jaw or both jaws can be movable relative to each other. One or both of the first jaw 20 and the second jaw 22 can also be provided with a plurality of teeth 28 for grasping tissue therebetween.

The first opening 24 and the second opening 26 align relative to each other when in the clamped position, such that the suture can be passed through the aligned openings. The first opening 24 is shown as an oval opening, and the second opening 26 is shown as a circular hole that is in communication with a slot 28 extending from the hole to the perimeter of the second jaw 22. The slot 28 allows a suture which is passed through the tissue to be removed from the second jaw 22. It will be appreciated that the exact shape of the first opening 24 and second opening 26 can vary, depending on the application.

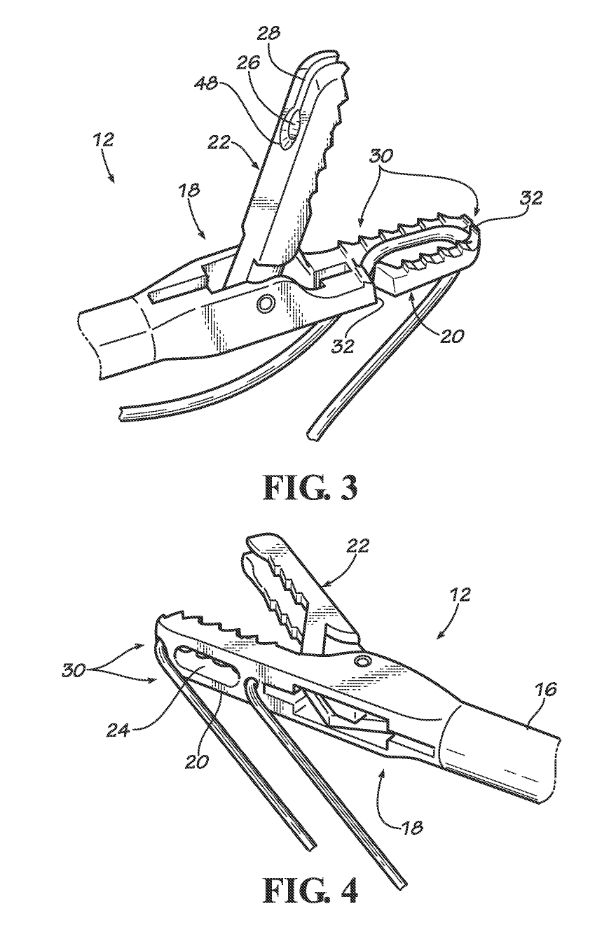

The first jaw 20 includes a suture positioner 30 for positioning a suture across the first opening 24. In the illustrated embodiment, the suture positioner 30 is in the form of a pair of slots 32 on generally opposite sides of the first opening 24. When the suture is positioned within the slots, as shown in FIGS. 3-5, the suture extends across the generally center of the first opening 24 so that it can be reliably and easily retrieved with the suture retriever 14 when in the clamped position, discussed below. The slots allow the suture to be manually loaded into the first jaw 20, but other types of suture positioners such as clips, protrusions, etc. could also be used.

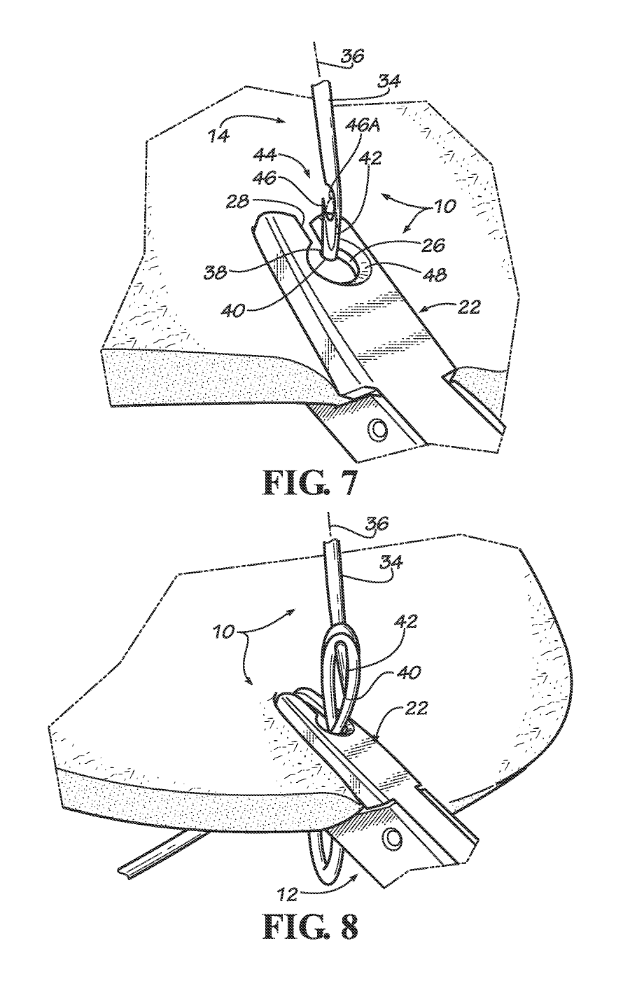

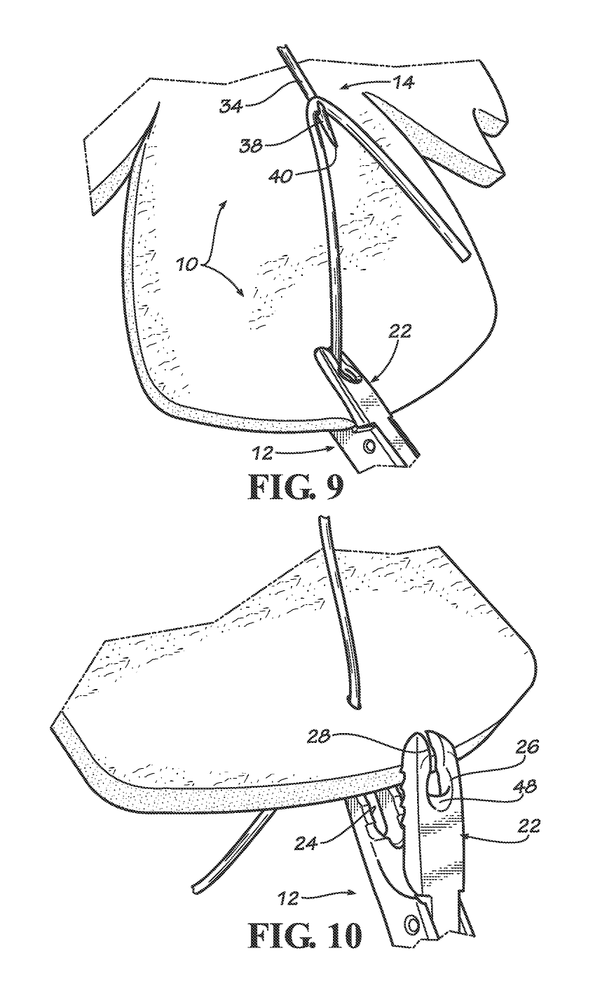

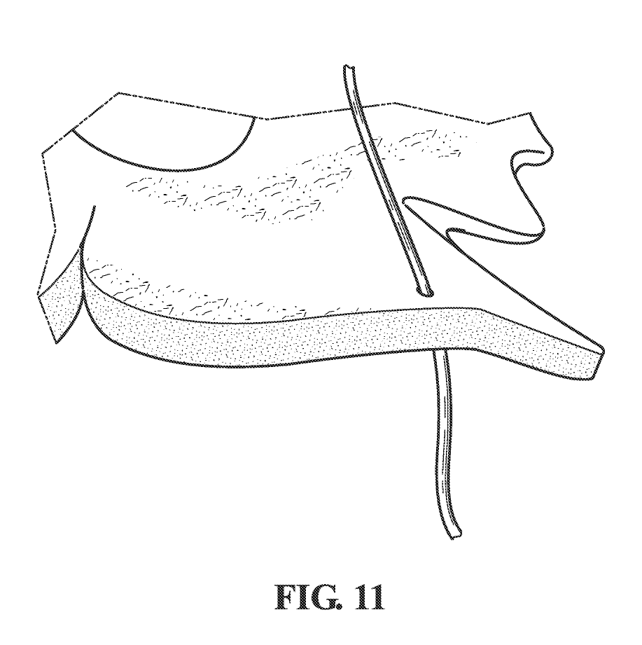

The suture retriever 14 includes an elongate rod 34 with a central axis 36 and a distal end 38. The distal end 38 includes a piercing member 40 for piercing tissue through which the suture is to be passed. In the embodiment shown, the distal end 38 includes a sleeve 42 with a sharpened distal edge (not separately numbered) defining the piercing member 40. The sleeve 42 can be tapered toward the distal end (as shown in FIGS. 7 and 9) or can be straight (i.e., cylindrical).

The suture retriever 14 also includes a hook 44 positioned proximally of the distal end 38. As the suture retriever 14 passes at least partially through the first opening 24 and the second opening 26, the hook 44 is used to retrieve the suture held across the first opening 24. To that end, the hook 44 includes a lip 46 which snags the suture as the suture retriever 14 is withdrawn from the tissue grasper 12. In the embodiment shown, the lip 46 is immovable relative to the elongate rod 34; however, the lip 46 can be movable toward and away from the central axis as indicated by the alternate position 46A shown in phantom lines. Alternatively, the hook 44 may be moved axially into the sleeve 42 to establish a grip on the suture.

During surgery, the tissue grasper 12 and suture retriever 14 can be packaged, sterilized and sold as a kit which is provided to a surgeon for surgery. The suture is first loaded into the first jaw 20 of the tissue grasper 12 (FIGS. 3-5). With the tissue grasper 12 clamped against the tissue at a desired location (FIG. 6), the piercing member 40 of the suture retriever 14 is positioned adjacent to the tissue on a side of the second jaw 22, opposite from the first jaw 20 holding the suture (FIG. 7). The piercing tip is moved into the tissue to pierce and form a hole for the suture in the tissue, and is the primary way of creating the hole in the tissue. The piercing member 40 mates with the second jaw 22 of the tissue grasper 12 to aim the suture retriever 14 at the suture. More particularly, the second opening 26 includes a beveled inlet edge 48 and the sleeve 42 is tapered for assisting with alignment of the suture retriever 14. The suture retriever 14 is inserted through the aligned openings 24 and 26 and the hook 44 is used to retrieve and withdraw the suture.

In the illustrated embodiment, the suture retriever 14 includes the piercing member 40. However, it is also possible that the tissue grasper 12 can be configured to include the piercing member 40. The hole in the tissue may be created by a feature which is integral with the tissue grasper 12, with the suture retriever 14 primarily following the path created by the hole. Moreover, the suture retriever 14 may be entirely incorporated into the second jaw 22, giving a surgeon the choice of shuttling the suture through the hole or bringing out the same cannula.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A suture passer for passing a suture through tissue during surgery, said suture passer comprising:
a tissue grasper having a first jaw with a first opening and a second jaw with a second opening, at least one of said first jaw and said second jaw being movable between an open position and a clamped position, said first opening and said second opening aligning relative to each other when in the clamped position, said first jaw having a suture positioner for positioning a suture across said first opening;
a piercing member; and
a suture retriever including a distal end and a hook positioned proximally of said distal end, said suture retriever passing at least partially through said first opening and said second opening and said hook being used to retrieve the suture held across said first opening.

2. The suture passer of claim 1, wherein said distal end of said suture retriever includes said piercing member.

3. The suture passer of claim 2, wherein said distal end of said suture retriever includes a sleeve with a sharpened distal edge defining said piercing member.

4. The suture passer of claim 3, wherein said sleeve is tapered toward said distal end.

5. The suture passer of claim 1, wherein said suture retriever includes an elongate rod with a central axis, and said hook includes a lip which is movable toward and away from said central axis.

6. The suture passer of claim 1, wherein said second opening includes a beveled inlet edge for assisting with alignment of said suture retriever.

7. The suture passer of claim 1, wherein said suture positioner of said first jaw includes a pair of slots on generally opposite sides of said first opening.

8. The suture passer of claim 1, wherein said second jaw includes a slot in communication with said second opening.

9. The suture passer of claim 1, wherein each of said first jaw and said second jaw have a plurality of teeth for grasping tissue therebetween.

10. The suture passer of claim 1, wherein said second jaw is movable toward and away from said first jaw.

11. A surgical kit for passing a suture through tissue during surgery, said suture passer kit comprising:
a tissue grasper having an elongate member with a first jaw and a second jaw positioned at a distal end of said elongate member, said first jaw having a first opening and said second jaw having a second opening, at least one of said first jaw and said second jaw being movable between an open position and a clamped position, said first opening and said second opening aligning relative to each other when in the clamped position, said first jaw having a suture positioner for positioning a suture across said first opening; and
a suture retriever including an elongate rod with a distal end, a piercing member at said distal end, and a hook positioned proximally of said piercing member.

12. The surgical kit of claim 11, wherein said distal end of said suture retriever includes a sleeve with a sharpened distal edge defining said piercing member.

13. The surgical kit of claim 12, wherein said sleeve is tapered toward said distal end.

14. The surgical kit of claim 11, wherein said elongate rod of said suture retriever includes a central axis, and said hook includes a lip which is movable toward and away from said central axis.

15. The surgical kit of claim 11, wherein said second opening includes a beveled inlet edge for assisting with alignment of said suture retriever.

16. The surgical kit of claim 11, wherein said suture positioner of said first jaw includes a pair of slots on generally opposite sides of said first opening.

17. The surgical kit of claim 11, wherein said second jaw includes a slot in communication with said second opening.

18. The surgical kit of claim 11, wherein each of said first jaw and said second jaw have a plurality of teeth for grasping tissue therebetween.

19. The surgical kit of claim 11, wherein said second jaw is movable toward and away from said first jaw.
